(19) United States
(12) Patent Application Publication
(10) Pub. No.: US 2025/0051806 A1
(43) Pub. Date: Feb. 13, 2025

(54) NOVEL PRETREATMENT FOR RECALCITRANT BIOMASS FOR BIOGAS GENERATION

(71) Applicant: SixRing Inc., Calgary (CA)

(72) Inventors: Corynne O'Farrell, Calgary (CA); Julie Greer, Calgary (CA); Markus Weissenberger, Calgary (CA); Alejandra Enriquez, Calgary (CA)

(21) Appl. No.: 18/437,615

(22) Filed: Feb. 9, 2024

(30) Foreign Application Priority Data
Aug. 11, 2023 (CA) 3209265

(51) Int. Cl. C12P 5/02

(52) U.S. Cl. CPC C12P 5/023

(57) ABSTRACT

In one example, a method includes providing a recalcitrant biomass; exposing said highly recalcitrant biomass to a delignification reaction wherein said delignification reaction comprising the exposure of said highly recalcitrant biomass to an acidic composition comprising a modified Caro's acid for a period of time sufficient to yield a treated cellulose which has a final lignin content of up to 100% less than the initial lignin content of the biomass; providing a digester whose contents comprises at least one inoculum comprising a microbial community capable of converting organic material into methane under anaerobic conditions; adding said treated cellulose to said digester; allowing sufficient time for the digester to degrade at least a portion of said treated cellulose and optionally, at least a portion of said organic material to yield a biogas composition comprising methane; capturing said biogas composition; and storing said biogas.

### RELATED APPLICATIONS

This application claims priority to Canadian Patent Application No. 3,209,265, titled “Novel Pretreatment For Recalcitrant Biomass For Biogas Generation,” filed on Aug. 11, 2023, which is hereby incorporated by reference in its entirety.

### TECHNICAL FIELD

The present invention is directed to the use of a delignification pretreatment on lignocellulosic biomass in the generation of biogas, more specifically, in the use of a substantially lignin-free cellulose as a replacement of a portion of the organic matter (feedstock) fed into an anaerobic digester.

### BACKGROUND

Biogases are the product of microbial degradation of various organic materials (both plant-based and animal-based products) in the absence of oxygen, i.e., in an anaerobic environment. This microbial degradation in an oxygen-free environment is called anaerobic digestion. Biogas can refer to gas produced naturally and industrially and generally contains between 45-75% methane (CH₄ or natural gas), 25-45% carbon dioxide (CO₂), and trace amounts of various other gases.

Natural sources of biogas include environments such as swamps, which generate methane by action of methanogens. A more common source of biogas is related to industrial activities related to waste disposal, principally related to landfills. A second, less widespread, source of biogas from human activities comes from anaerobic digesters and is used to generate methane and recycle organic waste to be used in fertilizing the field. Biogas is generated when organic waste is exposed to microbes, i.e., bacteria, archaea, and fungi, under anaerobic conditions (no oxygen present) to break down the organic compounds. This biodegradation yields gas (biogas), liquids and solids. The latter two, called digestate, can be used as soil amendments in fields for agriculture. The composition and nutrient content of the digestate is greatly impacted by the feedstock that undergoes anaerobic digestion as well as the operation conditions of the digester.

One of the end uses of industrially generated biogas is to burn it to provide a source of heat for buildings, boilers and perhaps even the biodigester. Biogas from landfills and digesters can also be refined to separate the methane (natural gas) from the other non-desirable constituents of biogas such as, but not limited to, carbon dioxide, water vapor, hydrogen sulfide, and others. This refining step yields renewable natural gas (RNG) which can be used as is, can be injected into an existing natural gas grid, or even used for vehicles powered by natural gas.

A significant benefit of biogas recovered from biodigesters or landfills is the reduction of usage of fossil fuels. Use of biogas generated from biodigesters or landfills can provide a clean source of power, which also happens to reduce the amount of methane released into the atmosphere. Since natural gas (methane) is a greenhouse gas which is, pound for pound, over 20 times more dangerous to the atmosphere that carbon dioxide over a 20-year period, it is desirable to reduce the release of this gas into the atmosphere. By controlling the release of this gas from its main industrial sources, it is possible to harness the energy it provides all the while providing a green alternative to power generation. Anaerobic digestion provides additional benefits to communities and the environment such as reducing odours, pathogens, and the risk of water pollution associated with manure coming from livestock and improving soil health.

The United States currently has over 2,000 biogas systems spread out across the country, but it has the potential to add at least another 10,000 additional plants and thus having a significant positive impact of the environment. In fact, proper harnessing of the potential of biogas in the United States alone would be the equivalent of removing the emissions of millions of cars yearly.

In addition to environmental benefits, the increased implementation of anaerobic digesters using various agricultural and food waste would inevitably reduce the costs associated with waste management if such were simply directed to landfills. Additional advantages of greater implementation of anaerobic digesters in the United States include the building of thousands of biogas systems, which would support hundreds of thousands of construction jobs and the resulting biodigester plants would employ several tens of thousands of people to operate them.

Wastewater treatment plants can have on-site anaerobic digesters to treat sewage sludge recovered during treatment. The solids are separated while the water is released and, more often than not, the methane generated is simply burnt into the atmosphere (i.e., it is flared) without benefiting of the energy this combustion generates. Only roughly two thirds of wastewater treatment plants in the United States that have anaerobic digesters actually use the biogas they generate.

### Biogas Feedstocks

Food waste in landfills is responsible for over 20% of the volume of waste present in U.S. landfills. This food waste is an important source of natural gas as it breaks down. While landfills may capture the resultant biogas, putting organic wastes in landfills does not enable operators to recover the nutrients generated from the source organic material such as fats, oils, and grease collected from the food service industry (added to an anaerobic digester to increase biogas production).

Livestock waste is another important source of methane. An average dairy cow weighing 1000 pounds produces approximately 80 pounds of manure per day, which is a non-negligible source of methane. It was assessed, in 2015, that livestock waste contributed to 10% of all methane emissions in the United States but that only 3% of all manure was actually being recycled in biodigester plants. This is a significant lost opportunity as well as environmentally careless.

Organic waste in landfills produces biogas, which is released into the atmosphere. Methane generated from landfills are ranked as the third largest source of such gas (by volume) related to human activities in the United States. Microorganisms present in landfills are similar to those found in anaerobic digestion, in that they are capable of breaking down organic materials and generating biogas. As methane is a potent greenhouse gas, it is desirable to capture these emissions to utilize them as a potential source of energy.

Crop residues are a source of organic material which can be processed in an anaerobic digester. These residues are meant to include, but not be limited to, stalks, straw, and plant trimmings. There are sufficient crop residues to leave a portion on the field in order to reduce the amount of soil erosion and harvest the remainder for biogas production. It is estimated that there is over 100 million tons of crop residues available which could be used in biogas systems. One drawback of crop residues is that they contain lignin, which is very poorly digested in biodigesters. Crop residues are typically mixed with a variety of other organic materials to generate biogas.

### Composition of Biogas

The composition of biogas is dependent on the feedstock, as well as the conditions of the biodigester such as temperature, pH, organic loading rate, etc. Typical biogas composition is made up of 45-75% methane, 25-45% carbon dioxide and 5% or less of various other gases.

Commercial scale anaerobic biodigesters face a number of different issues, with the most common being feedstock variability, low process efficiency, and lower product quality. These issues can be really significant for the asset owner or operator as it could not only translate to a non-economically favorable digester but could have considerable safety hazards to operators and the community (i.e., generation of offensive odours and/or generation of increased amounts of hydrogen sulfide, H₂S). Currently, there are a wide range of techniques available to anaerobic digester operators and owners to overcome these challenges. Pretreatment of highly recalcitrant feedstocks has been utilized to improve the hydrolysis rate of certain types of biomasses that are more difficult to degrade by the microbial community present in the anaerobic digester. These pretreatments include physical pretreatments (such as grinding the biomass, chipping, cavitation, mechanical refining, deflaking, dispersing and use of a Hollander beater, etc.), thermal pretreatments (categorized as hydrothermal, microwave, extrusion, torrefaction, steam explosion, and wet oxidation), and chemical pretreatments (which covers acidic, basic, redox reactions and ionic liquids), and combinations thereof. Other pretreatments which have been considered and tested include biological pretreatment; electrochemical pretreatment; and various combination pretreatments also referred to as hybrid approaches. However, in most cases the high capital cost, high consumption of energy and chemicals, low delignification efficiencies, and sophisticated operating conditions are the major factor hindering their full-scale application.

Lignocellulosic biomass is a widely available resource which can be used in biogas production. When lignin is present in the unprocessed biomass, or in a pulp after incomplete delignification, and it is used as part of the feedstock for a biodigester, it results in a reduction of the rate of hydrolysis of said feedstock by the microbial community. This is due to lignin being highly recalcitrant to biodegradation, especially under anaerobic conditions. It has been observed that there is an inverse relationship between the amount of lignin in plant biomass and the corresponding biomethane potential, in which lignin limits the bioavailability of the more readily degradable cellulose as they are tightly linked together. Lignin, and its corresponding dissolved lignin components, will accumulate as the microbial community preferentially targets the more readily biodegradable compounds, which will eventually limit certain microbial activities and hinder the efficiency of other processes required for adequate anaerobic digestion. As such, it is preferable to minimize the amount of lignin remaining in the feedstock when the latter is used in anaerobic digestion for the generation of biogas in order to maximize the methane yield.

In addition to that, in jurisdictions where waste lignocellulosic biomass is limited, operators and asset owners have had to resort to the use of very recalcitrant, hard-to-degrade feedstocks (i.e., higher lignin content, high cellulose crystallinity, lower cellulose surface area, etc.). These have proven to be more difficult to incorporate in anaerobic digesters, resulting in inefficient biogas generation. Lignin has a complex aromatic and highly branched structure and is therefore not favoured by microorganisms. This complex structure blocks access to the cellulose part of the biomass that would normally be readily degradable. Lignin is not an ideal source of carbon for biogas generation due to the numerous degradation reactions that must occur making it a time-consuming process that few organisms are capable of. Also, as lignin is biodegraded, it produces oxidants and phenolic compounds, which will act as anti-microbials, hindering other members of the microbial community present in anaerobic digesters.

In light of the above, it is clear that biogas production needs to increase in the coming years in order to reduce countries dependence on oil and fossil fuel-based products, especially given the fact that such biogas facilities can be readily implemented. However, in order to optimize biogas production from such biogas facilities it is desirable to be able to provide a more consistent biogas output which is rich in methane. The composition of the biogas generated at such facilities may vary during the year depending on the available feedstock which is used. In many areas, farmers having a biogas facility on their farms or close by provide some or all of the feedstock. However, some feedstock is not necessarily available year-round or scarce and thus, changing the composition of the feedstock over the year has a direct impact on the generation of biogas and the composition thereof.

Because of these factors, it is desirable to provide an alternative substrate which will replace or supplement at least a portion of the organic material (feedstock) being used in the production of biogas. Accordingly, there is a need to provide a method to delignify biomass to obtain a cellulose that is substantially free of lignin that can be used as part of the feedstock in combination with other organic materials to generate consistent methane-rich biogas.

### SUMMARY

According to a first aspect of the present invention, there is provided a method to increase methane production from an anaerobic digester, wherein said method comprises the addition of a substrate or component which is substantially-free of lignin that forms part of the feedstock portion.

The terms “substantially depleted of lignin”, “substantially free of lignin”, “substantially lignin-free”, “ultra-low lignin” and “substantially lignin-depleted” refer to a lignocellulosic component that has been pretreated and whose remaining lignin content is less than 5% of the amount of lignin present prior to the lignocellulosic biomass undergoing a delignification treatment.

Many different types of organic materials can be fed to anaerobic digesters. In general, and for the purposes of this invention, “feed” is referred to as the overall material added to the anaerobic digester or similar vessel at an established frequency (i.e., daily). The “feed” is typically comprised of two components. A first component, which is an inoculum comprising a microbial community capable of breaking down organic matter and producing biogas. Preferably, this can be in the form of manure (bovine, porcine, or equine) or in the form of sewage/wastewater sludge. The second component comprises of organic materials, referred to as “feedstock” herein. Preferred examples of organic materials are food by-products, agricultural and slaughterhouse waste products, fats, oils, greases, industrial organic residues, waste lignocellulosic biomass, etc.

According to a preferred embodiment of the present invention, the component is a cellulose which has been processed to be substantially free of lignin.

In accordance with the present invention, it is possible to increase the methane yield from an anaerobic digester by carefully adjusting the biomass composition. Preferably, one adjustment which can be made is to incorporate a component which is substantially free of lignin. Preferably, a source of cellulose which is free of lignin can be generated by treating lignocellulosic biomass with a modified Caro's acid as disclosed in Canadian patent applications 3,110,553; 3,110,555; and 3,110,558.

According to another aspect of the present invention, there is provided a process to make biogas, said process comprising the steps of:
providing a digester adapted to receive a feed and capture a biogas composition resulting from anaerobic digestion;
providing said feed comprising:
   a first component comprising at least one inoculum;
   a feedstock component comprising:
      a second component comprising at least one organic material preferably rich in nitrogen; and
      a third component comprising a biomass component which is substantially free of lignin in an amount that ranges between 5% w/w up to but not including 100% w/w of the feedstock;
         wherein said at least one inoculum capable of converting a portion of said at least one organic material into methane under anaerobic conditions;
adding said feed to said digester;
allowing sufficient time for the feedstock to be degraded wherein at least a portion of said third component and at least a portion of said second component is converted to a biogas composition comprising methane;
optionally, continuously adding said feed comprising said biomass component which is substantially free of lignin in an amount that ranges between 5% w/w up to but not including 100% w/w of the feedstock;
capturing said biogas composition; and
optionally, storing at least a portion of said biogas.

According to another aspect of the present invention, there is provided a process to make biogas, said process comprising the steps of:
providing a digester adapted to receive a feed and capture a biogas composition resulting from anaerobic digestion;
providing a feed comprising:
   a feedstock component comprising of:
      a biomass component which is substantially free of lignin in an amount that ranges between 5% w/w to 100% w/w of the feedstock;
      optionally, at least one other organic material preferably rich in nitrogen; and
   at least one inoculum capable of converting a portion of said feedstock into methane under anaerobic conditions;
adding said feed to said digester;
allowing sufficient time for the feed to be degraded wherein at least a portion of said biomass and optionally, at least a portion of said organic material to yield a biogas composition comprising methane;
capturing said biogas composition; and
optionally, storing at least a portion of said biogas.

According to yet another aspect of the present invention, there is provided a method to increase and stabilize the volume of methane produced from a biogas digester by using a substantially lignin-free cellulose as partial or full replacement to the feedstock used for biogas production.

According to a preferred embodiment of the present invention, said substantially lignin-free cellulose is a cellulose where there remains less than 10% of the amount of lignin prior to a lignocellulosic biomass being delignified. Preferably, said substantially lignin-free cellulose is a cellulose where there remains less than 5% of the amount of lignin prior to a lignocellulosic biomass being delignified. More preferably, said substantially lignin-free cellulose is a cellulose where there remains less than 2.5% of the amount of lignin prior to a lignocellulosic biomass being delignified. Even more preferably, said substantially lignin-free cellulose is a cellulose where there remains less than 1% of the amount of lignin prior to a lignocellulosic biomass being delignified.

According to a preferred embodiment of the present invention, said substantially lignin-free cellulose and is present in an amount ranging from 5% w/w to 100% w/w of the total organic feedstock added to the biodigester. Preferably, said substantially lignin-free cellulose and is present in an amount ranging from 10 to 50% w/w of the total organic feedstock added to the biodigester. More preferably, said substantially lignin-free cellulose and is present in an amount ranging from 12.5 to 40% w/w of the total organic feedstock added to the biodigester.

According to a preferred embodiment of the present invention, said substantially lignin-free cellulose is the result of the delignification of a recalcitrant biomass by exposure to a modified Caro's acid.

According to another aspect of the present invention, there is provided a process to make biogas, said process comprising the steps of:
providing a digester which comprises at least one organic material and at least one inoculum capable of converting a portion of said at least one organic material into methane under anaerobic conditions;
providing a treated biomass which is substantially free of lignin in a range from 5 to 100% w/w of the total organic feedstock portion of the feed;
adding said biomass to said digester;
allowing sufficient time for the digester to degrade at least a portion of said treated biomass and at least a portion of said organic material to yield a biogas composition comprising methane;
capturing said biogas composition; and
optionally, storing said biogas.

According to yet another aspect of the present invention, there is provided a process to increase the amount of methane produced from a biodigester, said method comprising the steps of:
providing a digester which comprises at least one organic material and at least one inoculum capable of converting a portion of said at least one organic material into methane under anaerobic conditions;
providing a treated biomass which is substantially free of lignin in a range from 5 to 100% w/w of the total organic feedstock portion of the feed;
adding said biomass to said digester;
allowing sufficient time for the digester to degrade at least a portion of said treated biomass and at least a portion of said organic material to yield a biogas composition comprising methane;
capturing said biogas composition; and
optionally, storing said biogas.

According to another aspect of the present invention, there is provided a use of a substantially lignin-free cellulose as partial or full replacement to organic waste intended for biogas production to increase the volume of methane produced from a biogas digester. Preferably, the substantially lignin-free cellulose stabilizes the volume of methane produced from a biogas digester.

According to another aspect of the present invention, there is provided a use of a substantially lignin-free cellulose as partial or full replacement to organic waste intended for biogas production to decrease the volume of carbon dioxide produced from a biogas digester.

According to another aspect of the present invention, there is provided a method of using recalcitrant biomass in the production of methane from a bio-digester, said method comprising the steps of:
providing a recalcitrant biomass wherein said biomass comprises a lignin content above 10% of the dry weight of said biomass;
exposing said highly recalcitrant biomass to a delignification reaction wherein said delignification reaction comprising the exposure of said highly recalcitrant biomass to an acidic composition comprising a modified Caro's acid for a period of time sufficient to yield a treated biomass which has a final lignin content of up to 100% less than the initial lignin content of the biomass;
providing a digester whose contents comprise at least one inoculum comprising a microbial community capable of converting organic material into methane under anaerobic conditions;
adding said treated biomass to said digester;
optionally, adding at least one organic material;
allowing sufficient time for the digester to degrade at least a portion of said treated biomass and optionally, at least a portion of said organic material to yield a biogas composition comprising methane;
capturing said biogas composition; and
storing said biogas.

According to a preferred embodiment of the present invention, a portion of the digester's contents is removed daily from said digester and a substantially equivalent replacement amount comprised of said inoculum, said treated biomass and optionally, said least one organic material is added to the digester. Preferably, said treated biomass comprises up to 100% of the equivalent feedstock replacement amount. Preferably, said treated biomass comprises from 5% to 100% of the equivalent feedstock replacement amount.

It will be understood by the person skilled in the art that biogas is generated after organic materials (plant and animal products) are broken down when exposed to bacteria, archaea and fungi, in an anoxic environment (i.e., under anaerobic conditions). This is also referred to as anaerobic digestion. Anaerobic digestion of organic material yields biogas and residual solids and liquids which is called the digestate. The digestate is rich in nutrients that were present in the original organic material but is now more readily available for plants and soil. The amount, composition, and nutrient content of the digestate is determined by the type of feedstock used in the decomposition of the organic matter added to the digester.

According to a preferred embodiment of the present invention, it is desirable, to balance out the nitrogen-rich material with cellulose-rich material in order to achieve a carbon to nitrogen ratio of the feed ranging preferably between 20:1 and 30:1 to obtain optimal methane generation. High nitrogen content organic materials also referred to as organic material rich in nitrogen include manure, crops and crop waste, grass trimmings, animal by-products, ground coffee, etc. Low nitrogen content organic material include bark, wood chips, sawdust, straw, etc.

According to a preferred embodiment of the present invention, there is provided a method to generate biogas which employs a substantially lignin-free cellulosic component obtained from the exposure of a lignocellulosic feedstock to a modified Caro's acid under substantially milder conditions than other conventionally employed pulping processes (such as Kraft pulping). This approach allows for a greener process across the board as the lignocellulosic feedstock does not divert food resources away from animals or humans, as well as uses a very low energy input delignification process.

According to a preferred embodiment of the present invention, the feedstock is a cellulose wherein said cellulose has a content of hemicellulose of less than 15%, preferably less than 10% and more preferably less than 5%, and a Kappa number of less than 10, more preferably less than 5, and even more preferably, less than 2.

Biogas comprises several gases including, but not limited to, methane, carbon dioxide, hydrogen sulfide, and volatile fatty acids. In most cases, the residual solids and liquids (“digestate”) can be used as fertilizer for soils.

### BRIEF DESCRIPTION OF THE FIGURES

Features and advantages of embodiments of the present application will become apparent from the following detailed description and the appended figures, in which:

FIG. 1 is a graphical depiction of the methane production of the samples as per Experiment #2; and

FIG. 2 is a graphical depiction of the methane production of the samples as per Experiment #3.

### DETAILED DESCRIPTION

One of the technical barriers to greater adoption of biogas as an alternative fuel source is related to infrastructural challenges. One of such challenges is the variance in the availability of the feedstock used in anaerobic digesters. It is known that various organic materials may be added to an anaerobic digester and produce biogas, however, what is not as well understood or appreciated is that there are several gases which are formed during such anaerobic digestion. The varying composition of the biogas is correlated to the feed (organic material) added for anaerobic digestion as well as the operating conditions and feed input. The availability of feedstock, being of organic nature, naturally varies throughout the year as based on the growing seasons. Moreover, the winter season is generally associated with a reduced biogas production which causes a number of problems.

According to a preferred embodiment of the present invention, there is provided a method to increase the methane production from an anaerobic digester.

It has been surprisingly found that when using a substantially lignin-free cellulose as a component or the main component of the feedstock into an anaerobic digester, the methane gas volume produced increases as compared to a non-delignified biomass or an insufficiently delignified substrate.

According to a preferred embodiment of the present invention, there is provided a method to increase and stabilize the volume of methane produced from a biogas digester by using a substantially lignin-free cellulose as part of or as the feedstock for biogas production. It has been surprisingly found that using a substantially lignin-free cellulose as part of or as the feedstock to anaerobic digestion can increase the methane gas volume produced.

According to a preferred embodiment of the present invention, the composition of gases comprises at least 60% methane to be used as an efficient source of energy.

Currently, most biomass fed to anaerobic digesters is not delignified and the presence of lignin causes inhibition of the biodegradation of said biomass by microorganisms present in the biodigester and consequently hinder optimal methane production from such units. Preferably, by using cellulose obtained from a delignification of lignocellulosic biomass using a modified Caro's acid, as a lesser component or the main component of the feedstock added to anaerobic digesters, the cellulose is in a more readily accessible form for the microorganisms in the biodigester.

According to a preferred embodiment of the present invention, the feedstock used in anaerobic digestion is a cellulose which has been processed to be substantially free of lignin.

Preferably, the addition of a substantially-free of lignin component allows for an increase in the generation of methane in an anaerobic digester when all or part of the feedstock is replaced with said substantially lignin-free cellulose. Preferably, said substantially lignin-free component is cellulose and is present in an amount ranging from 5% w/w to 100% w/w of the total feedstock amount added daily in the biodigester. Preferably, the cellulose is hydrated, in some cases the water may be up to 90% of the weight of the cellulose. According to a preferred embodiment, the cellulose is present in an amount ranging from 10 to 50% w/w of the total organic feedstock in the biodigester. According to a more preferred embodiment of the present invention, the cellulose is present in an amount ranging from 12.5 to 40% w/w of the total organic feedstock in the biodigester. According to yet another preferred embodiment, the cellulose is present in an amount ranging from 15% w/w to 30% of the total organic feedstock in the biodigester. Preferably, the amount of biomass component may be adjusted based on the composition of the organic content present in the digester. It is also desirable that given the possible fluctuations between various biodigesters (due to their different organic content and microbial communities), a pre-determination be done to assess the optimal concentration of the biomass component to be incorporated with the other organic content inside the digester so as not to incorporate an amount which is not optimal as the component may be more costly than the other organic content inside the biodigester.

It is generally accepted that biogas is formed mainly by the degradation of organic materials such as: carbohydrates, proteins, and lipids. The lignin fraction present in various feedstock added to digesters is known to be difficult to degrade by the microorganisms present in the digesters. While anaerobic digestion of lignin has been observed in certain environments, it requires a variety of microorganisms whose main pathway involves the enzymatic depolymerization of lignin. Even when these organisms are present, the process tends to be slow. Because of the complexity of the microbial community present in those systems, they are very difficult to reproduce on an industrial scale, making the applicability of the anaerobic digestion of lignin a complicated and difficult topic.

Recalcitrant biomass or highly recalcitrant biomass is another way of labelling such difficult to degrade biomass. The US Department of Energy has referred to biomass recalcitrance as being the resistance of plants to release their sugars for fermentation or upgrading. It is this recalcitrance which has been labelled to be the primary barrier to efficient and economical production of advanced biofuels. Types of recalcitrant biomass include but are not limited to: lignocellulosic biomass such as forestry by-products (such as wood shavings and the like); agricultural waste or by-products such as but not limited to, corn stover, rice straw, and wheat straw); paper industry waste; and other such as, but not limited to hemp, switch grass, etc. Within those categories, highly recalcitrant biomass is that containing large amounts of lignin, high crystallinity of the cellulosic portion or a high degree of polymerization of the cellulosic portion.

By adding a cellulose-rich component which is essentially devoid of lignin, it has been made possible to increase the generation of methane in an anaerobic digester. The delignification of biomass according to conventional pulping approaches typically results in pulp which is still high in lignin. Typically, further removal of the remaining lignin is performed through a bleaching process, which involves harsh chemicals and conditions that are not energy and environmentally conscious. To employ a bleached pulp in a biodigester would simply not be commercially viable or applicable on an industrial scale as it would be cost-prohibitive. In addition, any potential bleaching agent residues would negatively affect the digester health.

According to a preferred embodiment of the present invention, the biomass component is an unbleached cellulose. Preferably, the cellulose is obtained by the delignification of biomass through the exposure of such to a modified Caro's acid as per the following processes. A preferred embodiment of the process to delignify biomass, comprises the steps of:
providing a vessel;
providing biomass comprising lignin, hemicellulose and cellulose fibers into said vessel;
providing an aqueous acidic composition comprising an acid selected from the group consisting of: sulfuric acid; an alkylsulfonic acid; an arylsulfonic acid and combinations thereof;
providing a modifier component;
providing a peroxide component;
exposing said biomass to said acidic composition, said peroxide component and said modifier component to create a reaction mass for a period of time sufficient to a delignification reaction to occur and remove over 90 wt % of said lignin and hemicellulose from said biomass;
separating the dissolved lignin and hemicellulose form the solid cellulose;
neutralizing the solid cellulose to a neutral pH; and,
optionally, rinsing the cellulose to remove any acidic component remaining therein.

Preferably, the recalcitrant biomass comprises lignin, hemicellulose and cellulose fibers and is exposed to said acidic composition, said peroxide component and said modifier component which form a modified Caro's acid composition selected from the group consisting of: composition A; composition B and Composition C;
wherein said composition A comprises:
   sulfuric acid in an amount ranging from 20 to 70 wt % of the total weight of the composition;
   a modifier compound comprising an amine moiety and a sulfonic acid moiety selected from the group consisting of: taurine; taurine derivatives; and taurine-related compounds; and
   a peroxide;
wherein said composition B comprises:
   an alkylsulfonic acid; and
   a peroxide; wherein the acid is present in an amount ranging from 40 to 80 wt % of the total weight of the composition and where the peroxide is present in an amount ranging from 10 to 40 wt % of the total weight of the composition;
wherein said composition C comprises:
   sulfuric acid;
   a two-part modifier comprising:
      a compound comprising an amine moiety; and
      a compound comprising a sulfonic acid moiety; and
   a peroxide.

According to a preferred embodiment of the present invention, exposing said biomass to said modified Caro's acid composition will allow the delignification reaction to occur and remove over 90 wt % of said lignin and hemicellulose from said biomass.

Preferably, the delignification reaction is carried out at a temperature below 55° C. by a method selected from the group consisting of:
adding water into said vessel;
adding biomass into said vessel; and
using a heat exchanger.

Preferably, said sulfuric acid, said compound comprising an amine moiety and a sulfonic acid moiety and said peroxide are present in a molar ratio of no less than 1:1:1. Preferably, said sulfuric acid, said compound comprising an amine moiety and a sulfonic acid moiety are present in a molar ratio of no less than 1:1. Also preferably, said sulfuric acid, said compound comprising an amine moiety and a sulfonic acid moiety and said peroxide are present in a molar ratio of no more than 15:1:1. Preferably, said sulfuric acid, said compound comprising an amine moiety and a sulfonic acid moiety are present in a molar ratio of no more than 15:1. More preferably, said sulfuric acid, said compound comprising an amine moiety and a sulfonic acid moiety and said peroxide are present in a molar ratio of no less than 3:1:1. More preferably, said sulfuric acid, said compound comprising an amine moiety and a sulfonic acid moiety are present in a molar ratio of no less than 3:1.

According to a preferred embodiment of the method to delignify biomass as set out herein, said modifier compound comprises an amine moiety and a sulfonic acid moiety is selected from the group consisting of: taurine; taurine derivatives; and taurine-related compounds.

According to a preferred embodiment of the method to delignify biomass as set out herein, said taurine derivative or taurine-related compound is selected from the group consisting of: taurolidine; taurocholic acid; tauroselcholic acid; tauromustine; 5-taurinomethyluridine and 5-taurinomethyl-2-thiouridine; homotaurine (tramiprosate); acamprosate; and taurates; as well as aminoalkylsulfonic acids where the alkyl is selected from the group consisting of C₁-C₅ linear alkyl and C₁-C₅ branched alkyl. Preferably, said linear alkylaminosulfonic acid is selected form the group consisting of: methyl; ethyl (taurine); propyl; and butyl. Preferably, said branched aminoalkylsulfonic acid is selected from the group consisting of: isopropyl; isobutyl; and isopentyl.

According to a preferred embodiment of the present invention, said modifier compound comprising an amine moiety and a sulfonic acid moiety is taurine.

According to a preferred embodiment of the present invention, said sulfuric acid and compound comprising an amine moiety and a sulfonic acid moiety are present in a molar ratio of no less than 3:1.

According to a preferred embodiment of the present invention, said compound comprising an amine moiety is an alkanolamine is selected from the group consisting of: monoethanolamine; diethanolamine; triethanolamine; and combinations thereof.

According to a preferred embodiment of the present invention, said compound comprising a sulfonic acid moiety is selected from the group consisting of: alkylsulfonic acids and combinations thereof.

According to a preferred embodiment of the present invention, said alkylsulfonic acid is selected from the group consisting of: alkylsulfonic acids where the alkyl groups range from C₁-C₆ and are linear or branched; and combinations thereof.

According to a preferred embodiment of the present invention, said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof.

According to a preferred embodiment of the present invention, said alkylsulfonic acid; and said peroxide are present in a molar ratio of no less than 1:1.

According to a preferred embodiment of the present invention, said compound comprising a sulfonic acid moiety is methanesulfonic acid.

According to a preferred embodiment of the present invention, in Composition C, said sulfuric acid and said a compound comprising an amine moiety and said compound comprising a sulfonic acid moiety are present in a molar ratio of no less than 1:1:1.

According to a preferred embodiment of the present invention, in Composition C, said sulfuric acid, said compound comprising an amine moiety and said compound comprising a sulfonic acid moiety are present in a molar ratio ranging from 28:1:1 to 2:1:1.

According to preferred embodiment of the present invention, the modified Caro's acid (as disclosed in Canadian patent application 3,128,678) comprises: sulfuric acid; a heterocyclic compound; and wherein sulfuric acid and said a heterocyclic compound; are present in a molar ratio of no less than 1:1. Preferably, the sulfuric acid and said heterocyclic compound are present in a molar ratio ranging from 28:1 to 2:1 More preferably, the sulfuric acid and heterocyclic compound are present in a molar ratio ranging from 24:1 to 3:1. Preferably, the sulfuric acid and heterocyclic compound are present in a molar ratio ranging from 20:1 to 4:1. More preferably, the sulfuric acid and heterocyclic compound are present in a molar ratio ranging from 16:1 to 5:1. Preferably, the sulfuric acid and heterocyclic compound are present in a molar ratio ranging from 12:1 to 6:1. Also preferably, said heterocyclic compound has a molecular weight below 300 g/mol. Also preferably, said heterocyclic compound has a molecular weight below 150 g/mol. More preferably, said heterocyclic compound is a secondary amine. According to a preferred embodiment of the present invention, said heterocyclic compound is selected from the group consisting of: imidazole; triazole; and N-methylimidazole.

According to preferred embodiment of the present invention, the modified Caro's acid (as disclosed in Canadian patent application 3,128,677) comprises: sulfuric acid; a modifying agent comprising a compound containing an amine group; and wherein sulfuric acid and said compound containing an amine group; are present in a molar ratio of no less than 1:1. Preferably, the sulfuric acid and said compound containing an amine group are present in a molar ratio ranging from 28:1 to 2:1. More preferably, the sulfuric acid and compound containing an amine group are present in a molar ratio ranging from 24:1 to 3:1. Preferably, the sulfuric acid and compound containing an amine group are present in a molar ratio ranging from 20:1 to 4:1. More preferably, the sulfuric acid and compound containing an amine group are present in a molar ratio ranging from 16:1 to 5:1. Preferably, the sulfuric acid and compound containing an amine group are present in a molar ratio ranging from 12:1 to 6:1. According to a preferred embodiment of the present invention, the modifying agent is selected in the group consisting of: TEOA; MEOA; pyrrolidine; DEOA; ethylenediamine; diethylamine; triethylamine; morpholine; MEA-triazine; and combinations thereof. According to a more preferred embodiment of the present invention, the modifying agent is TEOA; MEOA; pyrrolidine; DEOA; ethylenediamine; triethylamine.

According to preferred embodiment of the present invention, the modified Caro's acid (as disclosed in Canadian patent application 3,128,676) comprises: sulfuric acid; a modifying agent comprising an alkanesulfonic acid; and wherein sulfuric acid and said alkanesulfonic acid are present in a molar ratio of no less than 1:1. Preferably, said alkanesulfonic acid is selected from the group consisting of: alkanesulfonic acids where the alkyl groups range from C1-C6 and are linear or branched; and combinations thereof. Preferably, said alkanesulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof. More preferably, said alkanesulfonic acid is methanesulfonic acid. Also preferably, said alkanesulfonic acid has a molecular weight below 300 g/mol. Also preferably, said alkanesulfonic acid has a molecular weight below 150 g/mol. Preferably, the sulfuric acid and said alkanesulfonic acid and are present in a molar ratio ranging from 28:1 to 2:1. More preferably, the sulfuric acid and alkanesulfonic acid are present in a molar ratio ranging from 24:1 to 3:1. Preferably, the sulfuric acid and alkanesulfonic acid are present in a molar ratio ranging from 20:1 to 4:1. More preferably, the sulfuric acid and alkanesulfonic acid are present in a molar ratio ranging from 16:1 to 5:1. According to a preferred embodiment of the present invention, the sulfuric acid and alkanesulfonic acid are present in a molar ratio ranging from 12:1 to 6:1.

According to preferred embodiment of the present invention, the modified Caro's acid (as disclosed in Canadian patent application 3,128,675) comprises: sulfuric acid; a substituted aromatic compound; and wherein sulfuric acid and said substituted aromatic compound; are present in a molar ratio of no less than 1:1. Preferably, the substituted aromatic compound comprises at least two substituents. More preferably, at least one substituent is an amine group and at least one of the other substituent is a sulfonic acid moiety. According to a preferred embodiment, the substituted aromatic compound comprises three or more substituent. According to a preferred embodiment of the present invention, the substituted aromatic compound comprises at least a sulfonic acid moiety. According to another preferred embodiment of the present invention, the substituted aromatic compound comprises an aromatic compound having a sulfonamide substituent, where the compound can be selected from the group consisting of: benzenesulfonamides; toluenesulfonamides; substituted benzenesulfonamides; and substituted toluenesulfonamides. Preferably, the sulfuric acid and said substituted aromatic compound and are present in a molar ratio ranging from 28:1 to 2:1. More preferably, the sulfuric acid and substituted aromatic compound are present in a molar ratio ranging from 24:1 to 3:1. Preferably, the sulfuric acid and substituted aromatic compound are present in a molar ratio ranging from 20:1 to 4:1. More preferably, the sulfuric acid and substituted aromatic compound are present in a molar ratio ranging from 16:1 to 5:1. Preferably, the sulfuric acid and substituted aromatic compound are present in a molar ratio ranging from 12:1 to 6:1.

According to preferred embodiment of the present invention, the modified Caro's acid (as disclosed in Canadian patent application 3,128,674) comprises: sulfuric acid; a modifying agent comprising an arylsulfonic acid; and optionally, a compound containing an amine group; wherein sulfuric acid and said a arylsulfonic acid; are present in a molar ratio of no less than 1:1. Preferably, the compound containing an amine group is selected from the group consisting of: imidazole; N-methylimidazole; triazole; monoethanolamine (MEOA); diethanolamine (DEOA); triethanolamine (TEOA); pyrrolidine and combinations thereof. According to a preferred embodiment of the present invention, sulfuric acid and the peroxide are present in a molar ratio of approximately 1:1. Preferably, the sulfuric acid and said arylsulfonic acid and are present in a molar ratio ranging from 28:1 to 2:1. More preferably, the sulfuric acid and arylsulfonic acid are present in a molar ratio ranging from 24:1 to 3:1. Preferably, the sulfuric acid and arylsulfonic acid are present in a molar ratio ranging from 20:1 to 4:1. More preferably, the sulfuric acid and arylsulfonic acid are present in a molar ratio ranging from 16:1 to 5:1. According to a preferred embodiment of the present invention, the sulfuric acid and arylsulfonic acid are present in a molar ratio ranging from 12:1 to 6:1. Also preferably, said arylsulfonic acid has a molecular weight below 300 g/mol. Also preferably, said arylsulfonic acid has a molecular weight below 150 g/mol. Even more preferably, said arylsulfonic acid is selected from the group consisting of: orthanilic acid; metanilic acid; sulfanilic acid; toluenesulfonic acid; benzenesulfonic acid; and combinations thereof.

According to preferred embodiment of the present invention, the modified Caro's acid (as disclosed in Canadian patent application 3,128,673) comprises: sulfuric acid; a heterocyclic compound; an alkanesulfonic acid; and wherein sulfuric acid and said a heterocyclic compound; are present in a molar ratio of no less than 1:1. Preferably, said aqueous acidic composition comprising: sulfuric acid; a heterocyclic compound; an arylsulfonic acid; and wherein sulfuric acid and said a heterocyclic compound; are present in a molar ratio of no less than 1:1. Preferably, the arylsulfonic acid is toluenesulfonic acid.

Preferably, the sulfuric acid, the heterocyclic compound and the alkanesulfonic acid are present in a molar ratio ranging from 28:1:1 to 2:1:1. More preferably, the sulfuric acid the heterocyclic compound and the alkanesulfonic acid are present in a molar ratio ranging from 24:1:1 to 3:1:1. Preferably, the sulfuric acid, the heterocyclic compound and the alkanesulfonic acid are present in a molar ratio ranging from 20:1:1 to 4:1:1. More preferably, the sulfuric acid, the heterocyclic compound and the alkanesulfonic acid are present in a molar ratio ranging from 16:1:1 to 5:1:1. According to a preferred embodiment of the present invention, the sulfuric acid and heterocyclic compound are present in a molar ratio ranging from 12:1:1 to 6:1:1. Also preferably, said heterocyclic compound has a molecular weight below 300 g/mol. Also preferably, said heterocyclic compound has a molecular weight below 150 g/mol. Even more preferably, said heterocyclic compound is selected from the group consisting of: imidazole; triazole; n-methylimidazole; and combinations thereof. Preferably, the alkanesulfonic acid is selected from the group consisting of:

alkylsulfonic acids where the alkyl groups range from C1-C6 and are linear or branched; and combinations thereof. Preferably, said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof. More preferably, said alkylsulfonic acid is methanesulfonic acid.

According to preferred embodiment of the present invention, the modified Caro's acid (as disclosed in Canadian patent application 3,128,672) comprises: sulfuric acid; a carbonyl-containing nitrogenous base compound; and wherein sulfuric acid and said a carbonyl-containing nitrogenous base compound; are present in a molar ratio of no less than 1:1. According to a preferred embodiment of the present invention, the carbonyl-containing nitrogenous base compound is selected from the group consisting of: caffeine; lysine; creatine; glutamine; creatinine; 4-aminobenzoic acid; glycine; NMP (N-methyl-2-pyrrolidinone); histidine; DMA (N,N-dimethylacetamide); arginine; 2,3-pyridinedicarboxylic acid; hydantoin; and combinations thereof. Preferably, the sulfuric acid and said carbonyl-containing nitrogenous base compound and are present in a molar ratio ranging from 28:1 to 2:1. More preferably, the sulfuric acid and carbonyl-containing nitrogenous base compound are present in a molar ratio ranging from 24:1 to 3:1. Preferably, the sulfuric acid and carbonyl-containing nitrogenous base compound are present in a molar ratio ranging from 20:1 to 4:1. More preferably, the sulfuric acid and carbonyl-containing nitrogenous base compound are present in a molar ratio ranging from 16:1 to 5:1. According to a preferred embodiment of the present invention, the sulfuric acid and carbonyl-containing nitrogenous base compound are present in a molar ratio ranging from 12:1 to 6:1.

According to a preferred embodiment of the present invention, the biomass additive is a cellulose wherein said cellulose has a content of hemicellulose of less than 15%, preferably less than 10% and more preferably less than 5%, and a Kappa number of less than 10, more preferably less than 5, and even more preferably, less than 2.

### Lab Scale Testing

Serum bottles were used to conduct anaerobic digestion experiments. The serum bottles were set up by adding manure, digestate from a commercial scale digester and in some cases, feedstock comprised of agricultural waste and/or cellulose obtained via a delignification process involving the use of a modified Caro's acid as described earlier. Biogas production and methane concentrations were regularly measured throughout the experiments.

Experiment #1—Determination of Theoretical Methane Potential (TMP) of Canola Straw Biomass and a Substantially-Free of Lignin Cellulose Derived from Canola Straw

The purpose of this experiment was to determine the TMP of canola straw biomass and compare it with the TMP of a substantially-free of lignin cellulose derived from canola straw, whose lignin has been removed using the process described herein above. Canola straw biomass was milled to 1 mm in size and the substantially lignin-free cellulose was a ‘wet’ or ‘never-dried’ substrate, with a total solids content of 17% wt. These substrates were then suspended in water and analyzed for Chemical Oxygen Demand (COD) determination. COD is a measure of the amount of oxygen that can be consumed by reactions during the decomposition of organic matter suspended in water. This value describes how much energy is available in the organic matter and can be used to calculate the theoretical amount of methane that could be produced from the complete decomposition of that organic matter. The same samples were characterized by testing their total solids (TS) as well as volatile solids (VS). This was performed as per US EPA method 1684. The percentage of volatile solids is a measure of the amount of material lost after a sample is ignited. The results of these tests are reported in Table 1. Calculated based on COD, TS, and VS for Experiment #1. The COD results are the average of two determinations while TS and VS are performed in triplicate.

TABLE 1
Theoretical methane potential for canola straw biomass and substantially-
free of lignin cellulose derived from canola straw.

| | Chemical | | Theoretical |
|---|---|---|---|
| | Oxygen | | Methane |
| | Demand | Volatile | Potential |
| Substrate | (mg/L) | Solids (%) | (mL/g VS) |
| Canola Cellulose | 32850 | 16.86 | 675 |
| Canola Straw Biomass | 17750 | 81.36 | 151 |

The results of this experiment demonstrate the TMP of the substantially lignin-free cellulose to be over 4.5 times greater than that of the biomass from which it was derived. This is indicative that the amount of methane that can be generated from this cellulose would be 4.5 times higher than that of a canola straw biomass when degraded in an anaerobic digestion system, when the same amounts were added.

Experiment #2-Determination of the Biochemical Methane Potential (BMP) of Canola Straw Biomass and a Substantially-Free of Lignin Cellulose Derived from Canola Straw.

The purpose of this experiment was to determine and compare the BMP of canola straw biomass and compare it with the BMP of a substantially-free of lignin cellulose derived from canola straw, whose lignin has been removed using a modified Caro's acid as per a process as described herein above. The lignin-depleted cellulose obtained from said process is subsequently neutralized using NaOH and rinsed at least once.

This experiment was designed to corroborate the theoretical results obtained from Experiment #1. For the experiment preparation, the Total Solids (TS) and Volatile Solids (VS) were measured for both substrates, as well as the inoculum (digestate). Inoculum was prepared by incubating it under anaerobic conditions for three days prior to setting up the serum bottles. Substrates were added to serum bottles containing the prepared inoculum in an inoculum-to-substrate ratio ranging from 0.35 to 2.0, calculated based of the volatile solids of the materials. Corresponding substrate-free blank bottles were prepared by adding water in place of the substrate. Bottles were incubated at 35° C. for the duration of the experiment. Biogas and methane production of test samples were measured three times a week or as needed.

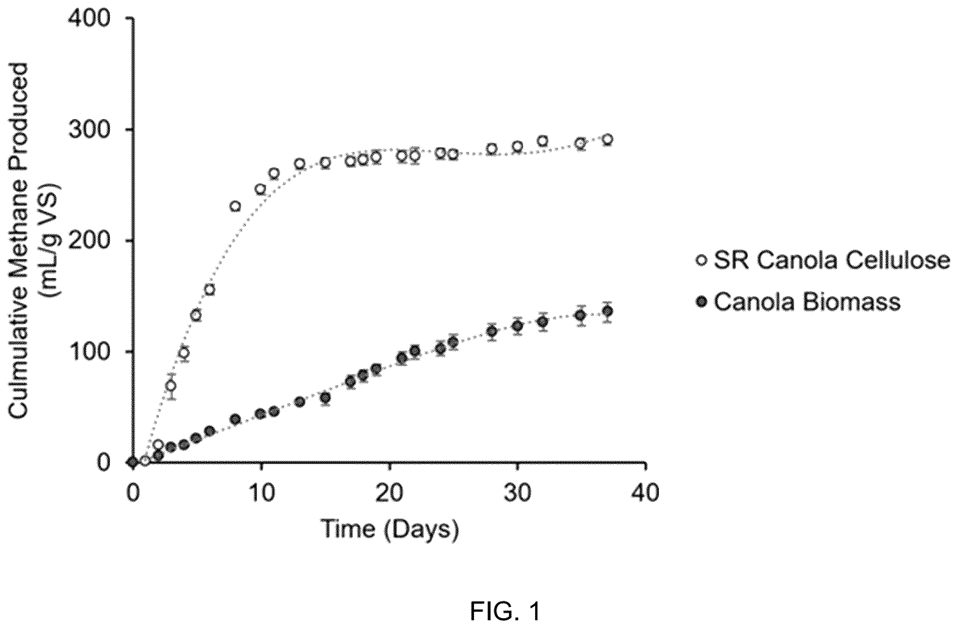

For analysis, the methane measurements were normalized to Standard Temperature Pressure (STP) conditions (101.35 kPa, 0° C.) and the methane generated from the substrate-free blanks was subtracted from the methane generated from the experimental substrates. This was done to ensure that the results are a reflection of the amount of methane produced from the addition of the substrate alone. The results for this experiment are shown in Table 2 and FIG. 1.

TABLE 2
Maximum Biochemical Methane Potential (from
FIG. 1) as well as corresponding lignin content
of the substrates tested in Experiment #2

| Substrate | BMP (mL CH₄/g VS) | Lignin content (%) |
|---|---|---|
| Canola Cellulose | 290 | 0.4 |
| Canola Straw Biomass | 136 | 17.9 |

This experiment has substantiated the advantage of using the herein described delignification process to pretreat a biomass prior to the use thereof in anaerobic digestion in order to increase methane production in anaerobic digestion.

In the biodigester, after a period of approximately 15 days, the methane production of the canola cellulose reached a stationary phase, in which the production of methane from the substrate had been maximized. The canola biomass (untreated), however, did not reach this stationary phase of production until after approximately 30 days of incubation. The cumulative volume of methane produced per gram of VS of the canola cellulose was more than double that of the canola straw biomass.

Moreover, another advantage of a preferred embodiment of the present invention is that it greatly reduces the amount of lignin present within the anaerobic digester so that as to avoid the accumulation of a substantial amount of lignin. Once the organic material is degraded in the anaerobic digester and the biogases are formed and released, lignocellulosic biomass materials will leave all but the recalcitrant lignin behind. After several additions of lignocellulosic biomass, the production of methane by the methanogenic organisms present in the anaerobic digester will begin to decline, as the organisms will be inhibited by the ever-increasing amounts of lignin in the system. According to a preferred embodiment of the present invention, the addition of ultra-low lignin content materials will not encounter this problem since the cellulosic material added daily will never contain sufficient amount of lignin to cause such difficulties to a biodigester.

Accordingly, there is proposed a method to increase both the volume of methane produced but also the speed at which it is generated in an anaerobic digester. Preferably, the process stabilized methane production from an anaerobic digester by providing the latter with a cellulose-rich and lignin depleted feedstock which overcomes the problem of lignin accumulation in the system.

### Experiment #3-Determination of the Effect of the Addition of a Substantially Lignin-Free Cellulose as a Component of the Feedstock

For this batch, the basic proof-of-concept was tested to confirm if the addition of a cellulose substantially free of lignin in a range between 25 to 100% w/w of the feedstock content increases methane generation in an anaerobic digestion system. Said cellulose is obtained by the delignification process referred to herein where the cellulose is removed from the reaction mixture and separated from the solubilized lignin and hemicellulose as a slurry of 8.5% w/w of solids. The objective of this batch was to test the effect of the substantially lignin-free cellulose addition as well as the optimal concentration of cellulose in the feedstock. A comparison was also carried out against a non-refined, non-delignified cellulose source (straw). The following samples were prepared:
1. Agricultural waste (no straw or substantially lignin-free cellulose)
2. 25% w/w of the feedstock as wheat straw (approx. 15% lignin)
3. 75% w/w of the feedstock as cellulose
4. 100% w/w of the feedstock as cellulose

Each sample contained 50% manure, 30% digestate, and 20% feedstock. The feedstock used was a combination of potato waste, slaughterhouse floatation screenings, and other various agricultural wastes with a carbon to nitrogen ratio (C:N)<20. In samples containing cellulose, the feedstock was replaced with the respective cellulose or dried wheat straw. The cellulose substantially free of lignin is a reference to the cellulose obtained from a delignification process as disclosed herein using a modified Caro's acid.

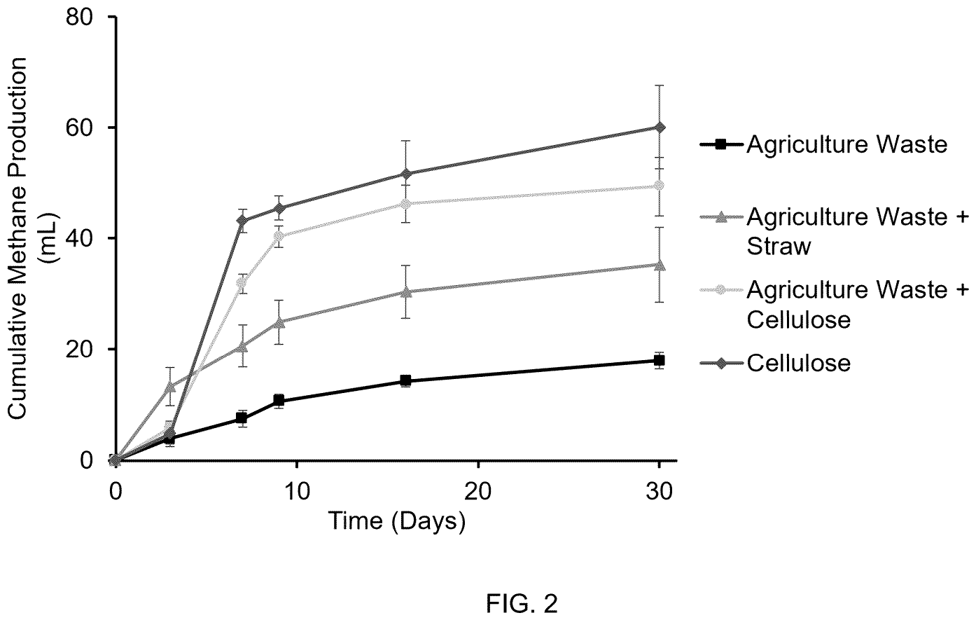

Generation of methane is initially greater in the samples containing straw (FIG. 2). However, just after 7 days, a sharp increase in methane production was observed for the samples containing cellulose, with the sample replacing 100% of the feed with cellulose producing the most methane. The control containing no cellulose or straw was consistently lower in volume of methane than all samples containing some form of cellulose (FIG. 2).

While the foregoing invention has been described in some detail for purposes of clarity and understanding, it will be appreciated by those skilled in the relevant arts, once they have been made familiar with this disclosure that various changes in form and detail can be made without departing from the true scope of the invention in the appended claims.

1. A process to make biogas, said process comprising:
providing a digester adapted to receive a feed and capture a biogas composition resulting from anaerobic digestion;
providing said feed comprising:
a first component comprising at least one inoculum;
a feedstock component comprising:
a second component comprising at least one organic material preferably rich in nitrogen; and
a third component comprising a biomass component which is substantially free of lignin in an amount that ranges between 5% w/w up to but not including 100% w/w of the feedstock,
wherein said at least one inoculum capable of converting a portion of said at least one organic material into methane under anaerobic conditions;
adding said feed to said digester;
allowing sufficient time for the feedstock to be degraded wherein at least a portion of said third component and at least a portion of said second component is converted to a biogas composition comprising methane;
optionally, continuously adding said feed comprising said biomass component which is substantially free of lignin in an amount that ranges between 5% w/w up to but not including 100% w/w of the feedstock;
capturing said biogas composition; and
optionally, storing at least a portion of said biogas.

2. The method of claim 1, further comprising:
continuously adding said feed comprising said biomass component which is substantially free of lignin in an amount that ranges between 5% w/w up to but not including 100% w/w of the feedstock; and
storing at least a portion of said biogas.

3. A method to increase and stabilize the volume of methane produced from a biogas digester by using a substantially lignin-free cellulose as partial or full replacement to the feedstock used for biogas production.

4. The method according to claim 3, wherein said substantially lignin-free cellulose is a cellulose where there remains less than 10% of the amount of lignin prior to a lignocellulosic biomass being delignified.

5. The method according to claim 3, wherein said substantially lignin-free cellulose is a cellulose where there remains less than 5% of the amount of lignin prior to a lignocellulosic biomass being delignified.

6. The method according to claim 3, wherein said substantially lignin-free cellulose is a cellulose where there remains less than 2.5% of the amount of lignin prior to a lignocellulosic biomass being delignified.

7. The method according to claim 3, wherein said substantially lignin-free cellulose is a cellulose where there remains less than 1% of the amount of lignin prior to a lignocellulosic biomass being delignified.

8. The method according to claim 3, wherein said substantially lignin-free cellulose and is present in an amount ranging from 5% w/w to 100% w/w of the total organic feedstock added to the biodigester.

9. The method according to claim 3, wherein said substantially lignin-free cellulose and is present in an amount ranging from 10 to 50% w/w of the total organic feedstock added to the biodigester.

10. The method according to claim 3, wherein said substantially lignin-free cellulose and is present in an amount ranging from 12.5 to 40% w/w of the total organic feedstock added to the biodigester.

11. The method according to claim 3, wherein said substantially lignin-free cellulose is the result of the delignification of a recalcitrant biomass by exposure to a modified Caro's acid.

12. A process to increase the amount of methane produced from a biodigester, said method comprising:
providing a digester which comprises at least one organic material and at least one inoculum capable of converting a portion of said at least one organic material into methane under anaerobic conditions;
providing a treated biomass which is substantially free of lignin in a range from 5 to 100% w/w of the total organic feedstock portion of the feed;
adding said biomass to said digester;
allowing sufficient time for the digester to degrade at least a portion of said treated biomass and at least a portion of said organic material to yield a biogas composition comprising methane;
capturing said biogas composition; and
optionally, storing said biogas.

13. The process of claim 12, further comprising storing said biogas.

14. A method of using recalcitrant biomass in the production of methane from a bio-digester, said method comprising:
providing a recalcitrant biomass wherein said biomass comprises a lignin content above 10% of the dry weight of said biomass;
exposing said highly recalcitrant biomass to a delignification reaction wherein said delignification reaction comprising the exposure of said highly recalcitrant biomass to an acidic composition comprising a modified Caro's acid for a period of time sufficient to yield a treated biomass which has a final lignin content of up to 100% less than the initial lignin content of the biomass;
providing a digester whose contents comprise at least one inoculum comprising a microbial community capable of converting organic material into methane under anaerobic conditions;
adding said treated biomass to said digester;
optionally, adding at least one organic material;
allowing sufficient time for the digester to degrade at least a portion of said treated biomass and optionally, at least a portion of said organic material to yield a biogas composition comprising methane;
capturing said biogas composition; and
storing said biogas.

15. The method according to claim 14, wherein a portion of the digester's contents is removed daily from said digester and a substantially equivalent replacement amount comprised of said inoculum, said treated biomass and optionally, said least one organic material is added to the digester.

16. The method according to claim 14, wherein said treated biomass comprises up to 100% of the equivalent feedstock replacement amount.

17. The method according to claim 14, wherein said treated biomass comprises from 5% to 100% of the equivalent feedstock replacement amount.

18. The method according to claim 14, further comprising:
adding at least one organic material.

19. The method according to claim 14, further comprising:
adding at least one organic material; and
allowing sufficient time for the digester to degrade at least a portion of said organic material to yield a biogas composition comprising methane.
